(19) United States
(12) Patent Application Publication
(10) Pub. No.: US 2015/0057344 A1
(43) Pub. Date: Feb. 26, 2015

(54) PELTATIN FOR THE TREATMENT OF CHRONIC INFLAMMATORY DISORDERS

(71) Applicant: NESTEC S.A., Vevey (CH)

(72) Inventors: Mohamed Nabil Bosco, Epalinges (CH); Christian Darimont-Nicolau, Lausanne (CH); Jalil Benyacoub, Epalinges (CH)

(21) Appl. No.: 14/465,801

(22) Filed: Aug. 21, 2014

Related U.S. Application Data
(63) Continuation of application No. PCT/EP2013/053493, filed on Feb. 21, 2013.

(30) Foreign Application Priority Data
Feb. 24, 2012 (EP) 12156855.4

(51) Int. Cl. A61K 31/365; A23L 1/30; A61K 8/49; A61Q 19/00; A61K 36/29; A61K 36/23

(52) U.S. Cl. CPC A61K 31/365; A61K 36/29; A61K 36/23; A61K 8/4973; A61Q 19/00; A23L 1/3002; A23V 2002/00
USPC 514/463

(57) ABSTRACT

The present invention generally relates to health benefits of natural compounds. In particular, the present invention relates peltatins, a group of natural compounds that can be used to treat, prevent or alleviate chronic inflammatory disorders. For example, the present invention concerns a composition comprising at least one peltatin for use in the treatment or prevention of chronic inflammatory disorders.

### CROSS REFERENCE TO RELATED APPLICATIONS

This application is a continuation of PCT/EP2013/053493, filed Feb. 21, 2013, which application claims the benefit of priority of EP 12156855.4, filed Feb. 24, 2012, the entire contents of which are incorporated herein by reference.

### FIELD OF THE INVENTION

The present invention generally relates to health benefits of natural compounds. In particular, the present invention relates peltatins, a group of natural compounds that can be used to treat, prevent or alleviate chronic inflammatory disorders. For example, the present invention concerns a composition comprising at least one peltatin for use in the treatment or prevention of chronic inflammatory disorders.

### BACKGROUND OF THE INVENTION

Inflammation is the complex biological response of tissues to harmful stimuli, such as pathogens, damaged cells, or irritants. It is generally a protective attempt by the organism to remove the injurious stimuli as well as initiate the healing process for the tissue. However, non-appropriately regulated inflammation can lead to several diseases irrespective of the age of the subject.

Ageing is often associated with a dysregulation of the immune system, such as a noted decline in cell-mediated immune response concomitant with an increased humoral immune dysfunction (e.g. lower response to vaccine). Ageing is furthermore often associated with a status of low-grade inflammation. In particular many elderly subjects are at increased risk of infectious and non-infectious diseases that contribute to morbidity and mortality.

Unwanted inflammation can be treated by proper medication. However, medication may always result in unwanted side effects and often requires the supervision of medical personnel.

There is a need for an alternative composition that can be used to treat or prevent chronic inflammatory disorders.

The present inventors have addressed this need.

### SUMMARY OF THE INVENTION

Consequently, it was the objective of the present invention to provide the art with natural compounds that can be used in compositions, that are easy to prepare and to store and that can be administered—preferably on a day-to-day basis—safely and without unwanted side effects and that can be used to treat or prevent chronic inflammation.

The present inventors were surprised to see that they could achieve this objective by the subject matter of the independent claim. The subject matter of the dependant claims further develops the idea of the present invention.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O′D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; and E. M. Shevach and W. Strober, 1992 and periodic supplements, Current Protocols in Immunology, John Wiley & Sons, New York, N.Y. Each of these general texts is herein incorporated by reference.

The present inventors have found in screening assays that peltatins can effectively activate hPPARs, which makes them interesting candidates for, e,g., nutritional applications.

### DETAILED DESCRIPTION

The peroxisome proliferators-activated receptors (PPARα, γ and β/δ) are nuclear receptors that act as a transcription factor upon activation. These nuclear factors are expressed with distinct patterns in many cell types. They regulate the transcription and expression of key target genes with a wide range of effects on the immune response. There are a variety of potential endogenous or synthetic ligands that bind PPARs with varying affinities and specificities, resulting in transcriptional activation or repression of target genes.

PPARγ is expressed in major immune cell types and enterocytes and a lot of evidence suggests that this nuclear receptor is also an important regulator of the inflammatory response. Indeed, in several rodent models of inflammatory or autoimmune diseases, ligands for PPARγ (and to some extent other PPARs) provide health benefits. Similar to rodents, PPARγ activation might have therapeutic activity in human diseases as PPARγ synthetic ligand was recently reported to have therapeutic activity in human patients with ulcerative colitis as a first demonstration (Lewis J. D., et al., Am. J. Gastroenterol. 2001, 96(12): 3323-3328). Anti-inflammatory effects of PPARγ are mediated by unliganded or bound receptors. So far, a couple of mechanisms were described to explain PPARγ ligand anti-inflammatory action. Endogenous or synthetic ligands were shown to downregulate diverse components of the inflammatory response in epithelial, innate and adaptive immune cells such as proinflammatory cytokines, chemokines and costimulatory molecules. Ligand-bound PPARγ repress the expression of inflammatory response genes via a so-called ligand dependent transrepression mechanism.

Consequently, the present invention relates in part to a composition comprising at least one peltatin for use in the treatment or prevention of chronic inflammatory disorders.

There is also provided herein a method of treating or preventing a chronic inflammatory disorder in a subject comprising administering a composition comprising at least one peltatin to said subject.

The chronic inflammatory disorder may be one which is treated or prevented by activation of a hPPAR, e.g., PPARγ and/or PPARα. In one embodiment the disease is one which is treated or prevented by activation of both PPARγ and PPARα.

There is also provided herein the use of at least one peltatin as an agonist of a hPPAR, in particular PPARγ and/or PPARα.

There is also provided herein a method of activating a hPPAR in a subject comprising administering at least one peltatin to said subject. In one embodiment the method comprises activation of PPARγ. In another embodiment the method comprises activation of both PPARγ and PPARα.

The invention also relates to the use of at least one peltatin for the preparation of a composition to treat or prevent inflammatory disorders.

Peltatins are well known in the art. They belong to lignans and may be found in some plants in the rhizome and in roots, for example. Peltatins and their derivatives (e.g., glycosides, esters) may be converted by the gut flora and the inventors speculate that they might be bioavailable as enterolactones and/or enterodiols.

Any chronic inflammatory disorder may be treated or prevented by administering at least one peltatin.

The inventors currently believe that the general effectiveness against all chronic inflammatory disorders is due to the mechanism of action via activation of hPPARs.

The inflammatory disorders may have a genetic origin, for example.

Typical inflammatory disorders that can be treated or prevented in accordance with the present invention may be selected from the group consisting of chronic inflammations such as inflammatory bowel disease, Crohn's disease, ulcerative colitis, necrotizing enterocolitis, skin inflammation, inflammatory bowel syndrome, liver inflammation, systemic lupus, obesity-associated inflammation, or age-related low-grade inflammation.

The compositions of the present invention may also be used to alleviate inflammation. While inflammation often may be a secondary disorder or symptom resulting from another disease, it often represents a significant reason for discomfort, pain and unwellness. Alleviating such inflammation related symptoms may represent a significant relief for a person or animal suffering from inflammation based symptoms.

The composition of the present invention may be to be administered to humans or animals, for example pet animals, such as dogs, cats, birds, rabbits, or guinea pigs.

The composition of the present invention may in particular be to be administered orally, enterally, parenterally or topically. The compositions may be provided in any galenical form normally available for the selected mode of administration.

The composition of the present invention may be administered to any age group. For example, the composition of the present invention may be to be administered to teenagers, adults, or the elderly.

As age-related low-grade inflammation is a typical problem of the older population, the composition of the present invention may be to be administered to the elderly.

A subject is considered as “elderly” if it has surpassed the first half of its average expected lifespan in its country of origin, preferably, if it has surpassed the first two thirds of the average expected lifespan in its country of origin, more preferably if it has surpassed the first three quarters of the average expected lifespan in its country of origin, most preferred if it has surpassed the first four fifths of the average expected lifespan in its country of origin.

For example, the composition of the present invention may be to be administered to people of at least 45 years of age.

The composition of the present invention may also to be administered during the cold season, e.g., from autumn to spring.

It may also be consumed at any time. It may be preferred to consume the composition of the present invention in the morning, e.g., to be prepared for challenges during the day.

The composition may, e.g., be selected from the group consisting of food compositions, food products, drinks, pet food products, dairy products, nutritional formulas, infant formulas, powdered nutritional formulations to be reconstituted in milk or water, food additives, nutritional supplements, nutraceuticals, pharmaceutical compositions, food ingredients and/or cosmetic compositions.

The composition may be provided in the form of a shelf stable powder. To obtain shelf stability and to ensure viability of the probiotics the composition may be provided with a water activity smaller than 0.2, for example in the range of 0.19-0.05, preferably smaller than 0.15. Water activity is a measurement of the energy status of the water in a system. It is defined as the vapor pressure of water deriving from the powder/product divided by that of pure water at the same temperature; therefore, pure distilled water has a water activity of exactly one.

The compositions of the present invention may be cleansing, protective, treatment or care creams, skincare lotions, gels or foams, such as cleansing or disinfecting lotions, bath compositions or deodorant compositions.

As regards more particularly the compositions for external topical administration, they may be aqueous, aqueous-alcoholic or oily solutions, solutions or dispersions of the lotion or serum type, emulsions of liquid or semi-liquid consistency, of the milk type, obtained by dispersion of a fatty phase in an aqueous phase (0/W) or vice-versa (W/0), or suspensions or emulsions of soft, semi-solid or solid consistency, of the cream type, aqueous or anhydrous gels, microemulsions, microcapsules, microparticles, or vesicular dispersions of ionic and/or non-ionic type.

A topical composition according to the invention may advantageously be formulated in any galenical form that is suitable for haircare, especially in the form of a hair lotion, a shampoo, especially an antidandruff shampoo, a hair conditioner, a detangler, a hair cream or gel, a styling lacquer, a hairsetting lotion, a treating lotion, a dye composition (especially for oxidation dyeing) optionally in the form of a colouring shampoo, a hair-restructuring lotion, a permanent-waving composition, a lotion or gel for combating hair loss, an antiparasitic shampoo or a medicated shampoo, especially an anti-seborrhoea shampoo, a scalp care product, which is especially anti-irritant, anti-ageing or restructuring, or which activates the blood circulation.

When the composition of the invention is an emulsion, the proportion of the fatty phase may range from 5% to 80% by weight, and preferably from 10% to 50% by weight, relative to the total weight of the composition. The oils, the emulsifiers and the coemulsifiers used in the composition in emulsion form are chosen from those conventionally used in the cosmetics and/or dermatological field. The emulsifier and the coemulsifier may be present, in the composition, in a proportion ranging from 0.3% to 30% by weight, and preferably from 0.5% to 20% by weight, relative to the total weight of the composition.

When the composition of the invention is an oily gel or solution, the fatty phase may represent more than 90% of the total weight of the composition.

The galenic forms for topical administration may also contain adjuvants that are customary in the cosmetics, pharmaceutical and/or dermatological field, such as hydrophilic or lipophilic gelling agents, hydrophilic or lipophilic active agents, preservatives, antioxidants, solvents, fragrances, fillers, screens, odour absorbers and dyestuffs. The amounts of these various adjuvants are those conventionally used in the field under consideration, and are, for example, from 0.01% to 20% of the total weight of the composition. Depending on their nature, these adjuvants may be introduced into the fatty phase and/or into the aqueous phase.

As fatty substances that may be used in the invention, mention may be made of mineral oils such as, for example, hydrogenated polyisobutene and liquid petroleum jelly, plant oils such as, for example, a liquid fraction of shea butter, sunflower oil and apricot kernel oil, animal oils such as, for example, perhydrosqualene, synthetic oils, in particular Purcellin oil, isopropyl myristate and ethylhexyl palmitate, unsaturated fatty acids and fluoro oils such as, for example, perfluoropolyethers. Use may also be made of fatty alcohols, fatty acids such as, for example, stearic acid and such as, for example, waxes, in particular paraffin wax, carnauba wax and beeswax. Use may also be made of silicone compounds such as silicone oils and, for example, cyclomethicone and dimethicone, and silicone waxes, resins and gums.

As emulsifiers that may be used in the invention, mention may, for example, be made of glyceryl stearate, polysorbate 60, the mixture of cetylstearyl alcohol/oxyethylenated cetylstearyl alcohol comprising 33 mol of ethylene oxide, sold under the name Sinnowax AO® by the company Henkel, the mixture of PEG-6/PEG-32/glycol stearate sold under the name Tefose® 63 by the company Gattefosse, PPG-3 myristyl ether, silicone emulsifiers such as cetyl dimethicone copolyol and sorbitan monostearate or tristearate, PEG-40 stearate, or oxyethylenated sorbitan monostearate (20 EO).

As solvents that may be used in the invention, mention may be made of lower alcohols, especially ethanol and isopropanol, and propylene glycol.

The composition of the invention may also advantageously contain a spring and/or mineral water, in particular chosen from Vittel water, waters from the Vichy basin, and la Roche Posay water.

As hydrophilic gelling agents, mention may be made of carboxylic polymers such as carbomer, acrylic copolymers such as acrylate/alkyl acrylate copolymers, polyacrylamides, and in particular the mixture of polyacrylamide, Cβ-14 isoparaffin and Laureth-7 sold under the name Sepigel 305® by the company SEPPIC, polysaccharides, for instance derivatives such as hydroxyalkylcelluloses, and in particular hydroxypropylcellulose and hydroxyethylcellulose, natural gums such as guar gum, locust bean gum, carob and xanthan gum, and clays.

As lipophilic gelling agents, mention may be made of modified clays such as bentones, metal salts of fatty acids, such as aluminium stearates and hydrophobic silica, or else ethylcellulose and polyethylene.

The compositions according to the invention may also be solid preparations constituting cleansing soaps or bars.

They may also be used for the scalp in the form of solutions, creams, gels, emulsions or mousses, or alternatively in the form of aerosol compositions also containing a propellant under pressure.

In the case of oral use in accordance with the invention for oral administration, the use of an ingestible support or carrier is preferred. The ingestible support or carrier may be of diverse nature depending on the type of composition under consideration.

Milk, yogurt, cheese, fermented milks, milk-based fermented products, ice creams, cereal-based products or fermented cereal-based products, milk-based powders, infant and baby formulas, food products of confectionary, chocolate or cereal type, animal feed, in particular for domestic animals, tablets, gel capsules or lozenges, liquid bacterial suspensions, oral supplements in dry form and oral supplements in liquid form are especially suitable for use as ingestible support or carrier.

The composition according to the invention to be administered orally may be formulated for example in the form of coated tablets, gel capsules, gels, emulsions, tablets, capsules, hydrogels, food bars, compact or loose powders, liquid suspensions or solutions, confectionery products, fermented milks, fermented cheeses, chewing gum, toothpaste or spray solutions or food carriers.

Tablets or lozenges, oral supplements in dry form and oral supplements in liquid form are suitable for use as dietetic or pharmaceutical supports or food carriers.

The composition may be, for example, a food supplement, which may be formulated via the usual processes for in particular producing sugar-coated tablets, gel capsules, gels, emulsions, tablets, capsules and hydrogels allowing controlled release.

The formulating agents and excipients for oral compositions, and in particular for food supplements, are known in this field and will not be the subject of a detailed description herein.

Any peltatin may be used for the purpose of the present invention. The peltatin used in the present invention is an agonist of a peroxisome proliferator-activated receptor PPAR. The at least one peltatin may have the following core structure

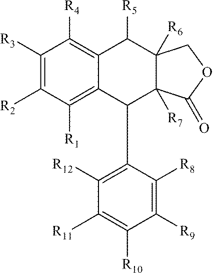

wherein R₁, R₂, R₃, R₄, R₈, R₉, R₁₀, R₁₁ and R₁₂ are independently selected from H, OH, OMe and O-sugar, or wherein adjacent R groups (e.g., R₁ and R₂, R₂ and R₃, R₃ and R₄, R₈ and R₉, R₉ and R₁₀, R₁₀ and R₁₁, or R₁₁ and R₁₂, preferably R₂ and R₃) together are —O—CH2-O— forming a cyclic 5-membered ring,
R₅ is selected from H, OH, OCO-alkyl wherein the alkyl is preferably C1 to C4 alkyl, O-alkyl wherein the alkyl is preferably C1-C4 alkyl, ═O, and O-sugar,
R₆ and R₇ are independently selected from H and OH.

The peltatin according to the present invention preferably has a structure such that R₄ is selected from OH, OMe or O-sugar, preferably OH. Preferably R5 is H.

In one embodiment, the peltatin contains no more than 2 hydroxy groups. In one embodiment, the peltatin contains one hydroxyl group.

The at least one peltatin preferably has the following stereochemistry:

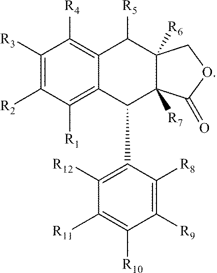

The at least one peltatin may be selected from the group consisting of α-peltatin or a derivative thereof or β-peltatin or a derivative thereof. The at least one pelatin may be a combination of α-peltatin or a derivative thereof and β-peltatin or a derivative thereof. For example, the at least one peltatin may be β-peltatin.

Typical peltatins that may be used in the framework of the present invention are depicted below:

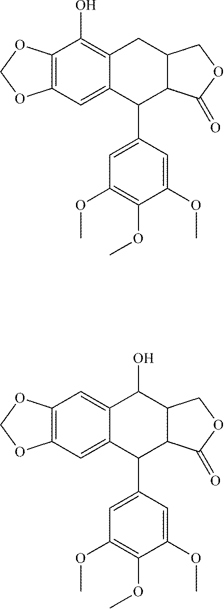 [SMILES: COc1cc(C2c3cc4c(c(O)c3CC3COC(=O)C32)OCO4)cc(OC)c1OC ; COc1cc(C2c3cc4c(cc3C(O)C3COC(=O)C23)OCO4)cc(OC)c1OC]

In a particularly preferred embodiment, peltatins that may be used in the framework of the present invention are depicted below:

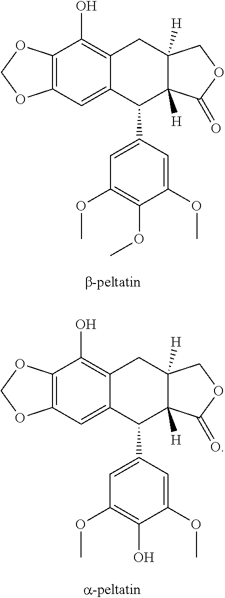 [SMILES: COc1cc([C@@H]2c3cc4c(c(O)c3C[C@H]3COC(=O)[C@@H]32)OCO4)cc(OC)c1OC ; COc1cc([C@@H]2c3cc4c(c(O)c3C[C@H]3COC(=O)[C@@H]32)OCO4)cc(OC)c1O]

The at least one peltatin may be provided as chemically pure compound. It may be synthesized chemically. It may also be provided as a plant extract, for example. A typical known plant sources of peltatins that may be used as source for the plant extract are may apple (Podophyllum peltatum), or cow parsley (Anthriscus sylvestris, for example.

Other possible plant sources for peltatins might be thymus, chestnuts, hazelnuts, chicory roots, flax seeds, sesame seeds, buckwheat seeds, or combinations thereof. These plant sources have the example, that they are generally approved for human or animal consumption, and hence food-grade.

Any extract may be used. For example, the extract may be a water extract, an alcoholic extract, and/or an extract with an organic solvent.

In one embodiment the extract is an alcoholic extract.

In a further embodiment the extract is a water extract.

The synthesis of peltatins is well known in the art. By way of example, the synthesis of peltatins is described in Masunari et al., Synthetic Communications 2001, 31(14), 2127-2136); and Yamaguchi et al., Chemical and Pharmaceutical Bulletin (Tokyo) 1984, 32, 1754-60.

Further synthetic methods are described in: Pelter et al., Tetrahedron Letters, 1985, 26(51), 6377-80; Takano et al., Heterocycles, 1987, 25(1), 69-73; Pelter et al., Journal of the Chemical Society, Perkin Transactions 1: Organic and Bio-Organic Chemistry, 1988, 1972-1999, (6), 1615-23; Ogiku et al, Bulletin of the Chemical Society of Japan, 1992, 65(12), 3495-7; Pelter et al., Journal of the Chemical Society, Perkin Transactions 1: Organic and Bio-Organic Chemistry, 1993, (21), 2621-9.

Assays for determining the hPPAR agonist activity of the peltatins described herein are well known in the art (see, for example, Forman et al., Cell. 1995 Dec. 1; 83(5):803-12; Han et al., Biol Pharm Bull. 2006 January; 29(1):110-3; Han et al., Diabetes. 2008 March; 57(3):737-45. Epub 2007 Dec. 7).

One such method involves utilizing a reporter gene construct wherein PPAR binding is assayed by measuring luciferase activity (see e.g., Forman et al., Cell. 1995 Dec. 1; 83(5):803-12; Han et al., Biol Pharm Bull. 2006 January; 29(1):110-3; Han et al., Diabetes. 2008 March; 57(3):737-45. Epub 2007 Dec. 7; US 2007244094).

In more detail, when PPARs fix their ligands they are able to shuttle from cytoplasm to the nucleus of HeLa cells. Then, PPARs heterodimerize with co-receptors called Retinoid-X-Receptors (RXR). Heterodimeric transcription factors PPAR/RXR are responsible for PPARs-mediated transcriptional program. In this system, hPPARs are fused to Gal4. Gal4 is a yeast transcription activator which specifically binds a Gal4 responsive element so-called Upstream Activation Sequence (UAS)— this short section in a promoter region strongly activates gene transcription. Therefore, cotransfection of Gal4-hPPARs with UAS-luciferase constructs allows identification hPPARs agonists. Agonists will stimulate luciferase transcription resulting in the formation of a functional enzyme that converts substrate to detectable signal by a chemiluminescent reaction.

An example of such a transfection assay method is described in US 2007244094 and comprises the following steps: HEK293 cells were grown in DMEM/F12 medium supplemented with 10% FBS and glutamine (Invitrogen) and incubated in a 5% CO₂ incubator at 37° C. The cells were co-transfected using DMRIE-C reagent (Invitrogen) in serum free medium (Opti-MEM, Invitrogen) with two mammalian expression plasmids, one containing the DNA sequence coding for the ligand binding domains of a PPAR fused to the yeast GAL4 DNA binding domain and the other containing the promoter sequence of the yeast GAL4 (UAS) fused to the firefly luciferase cDNA reporter. The next day, the medium was changed to DMEM/F12 medium supplemented with 5% charcoal treated serum (Hyclone) and glutamine. After 6 hrs the cells were trypsinized and seeded at a density of 50,000 cells/well into 96 well plates and incubated overnight as above. The cells were then treated with test compounds or vehicle and incubated for 18-24 hrs as above. Luciferase reporter activity was measured using the Steady-Glo Luciferase Assay Kit from Promega.

Preferably, the at least one peltatin used in the present invention has a maximum PPARγ activation activity of at least 70%, more preferably at least 100%, more preferably at least 120%, still more preferably at least 140% relative to the known PPARγ agonist Rosiglitazone. Preferably, the at least one peltatin has an AC50 with respect to PPARγ activation of less than 150 nM, more preferably less 100 nM, more preferably less than 80 nM. The agonist activity of the peltatin may be ascertained using the above described luciferase reporter gene assay.

Preferably, the at least one peltatin used in the present invention has a maximum PPARα activation activity of at least 70%, more preferably at least 100%, still more preferably at least 110% relative to the known PPARα agonist GW 9578. Preferably, the at least one peltatin has an AC50 with respect to PPARα activation of less than 100 nM, more preferably less 80 nM, still more preferably less 50 nM. The agonist activity of the peltatin may be ascertained using the above described luciferase reporter gene assay.

The GW 9578 referred to above is

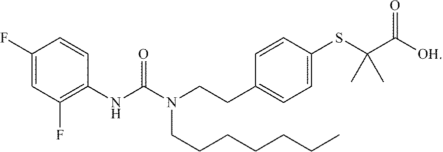 [SMILES: CCCCCCCN(CCc1ccc(SC(C)(C)C(=O)O)cc1)C(=O)Nc1ccc(F)cc1F]

GW 9578 is well known in the art and described, for example, in Brown et al., Journal of Medicinal Chemistry (1999), 42(19), 3785-3788.

Derivatives of α-peltatin and β-peltatin according to the present invention preferably have PPARγ and PPARα agonist activity that is substantially similar to, or greater than, that of α-peltatin and β-peltatin, respectively.

Preferably, the β-peltatin derivatives have a maximum PPARγ activity of at least 70%, more preferably at least 100%, still more preferably at least 120%, still more preferably at least 140% relative to the known PPARγ agonist Rosiglitazone. Preferably, the 3-peltatin derivatives used in the present invention have a maximum PPARα activation activity of at least 70%, more preferably at least 100%, still more preferably at least 110% relative to the known PPARα agonist GW 9578.

The effectiveness of the composition of the present invention follows a dose-response curve.

Any amount above a certain minimum level will achieve the object of the present invention, while larger amounts will produce more pronounced effects up to a saturation level.

In therapeutic applications, compositions are administered in an amount sufficient to at least partially cure or arrest the symptoms of the disease and its complications. An amount adequate to accomplish this is defined as “a therapeutically effective dose”. Amounts effective for this purpose will depend on a number of factors known to those of skill in the art such as the severity of the disease and the weight and general state of the patient.

In prophylactic applications, compositions according to the invention are administered to a patient susceptible to or otherwise at risk of a particular disease in an amount that is sufficient to at least partially reduce the risk of developing a disease. Such an amount is defined to be “a prophylactically effective dose”. Again, the precise amounts depend on a number of patient specific factors such as the patient's state of health and weight.

The compositions of the present invention may be administered in a therapeutically effective dose or a prophylactically effective dose.

For example the composition of the present invention may be to be administered in an amount corresponding to about 0.01 to 100 mg dry weight peltatins/kg body weight, e.g., about 0.05 to 50 mg dry weight peltatins/kg body weight, or about 1 to 20 mg dry weight peltatins/kg body weight.

To ensure regular intake of peltatins, the composition may be to be administered immediately before or during a meal. For example, peltatins may be an integral part of the meal, so that the composition of the present invention is administered with each meal.

To ensure a certain effectiveness of the composition of the present invention, the composition may contain a therapeutically effective dose or a prophylactically effective dose of the present invention per serving.

For example, the composition may comprise an amount of about 1-1000 mg peltatins per kg dry weight of the composition, e.g., an amount of about 5-200 mg peltatins per kg dry weight of the composition, or an amount of about 10-100 mg peltatins per kg dry weight of the composition.

Those skilled in the art will understand that they can freely combine all features of the present invention described herein, without departing from the scope of the invention as disclosed. In particular, features described for the composition of the present invention may be applied to the use of the present invention and vice versa.

Further advantages and features of the present invention are apparent from the following Examples and Figures.

### BRIEF DESCRIPTION OF THE DRAWINGS

FIG. 1 shows the principle of hPPAR bioassay used for screening. The assays detect binding of a ligand to the hPPAR of interest by directly measuring luciferase activity. When PPARs fixe their ligands they are able to shuttle from cytoplasm to the nucleus of HeLa cells. Then, PPARs heterodimerize with co-receptors called Retinoid-X-Receptors (RXR). Heterodimeric transcription factors PPAR/RXR are responsible for PPARs-mediated transcriptional program. In this system, hPPARs are fused to Gal4. Gal4 is a yeast transcription activator which specifically binds a Gal4 responsive element so-called Upstream Activation Sequence (UAS), this short section in a promoter region strongly activates gene transcription. Therefore, cotransfection of Gal4-hPPARs with UAS-luciferase constructs allows identification hPPARs agonists. Agonists will stimulate luciferase transcription resulting in the formation of a functional enzyme that converts substrate to detectable signal by a chemiluminescent reaction.

FIG. 2 shows dose-responses effects of beta-Peltatin used at different concentration (nM) for PPARα activation (black curve). The AC50 (upper part of the insert) and the maximum % activation (lower part of the insert) are given. Cellular toxicity was also assessed with standard method compared to untreated cells and shown in % of toxicity. As shown in the below grey curve no toxicity was observed even at highest dose of beta-Peltatin used.

FIG. 3 shows dose-responses effects of beta-Peltatin used at different concentration (nM) for PPARg activation (black curve). The AC50 (upper part of the insert) and the maximum % activation (lower part of the insert) are given. Cellular toxicity was also assessed with standard method compared to untreated cells and shown in % of toxicity. As shown in the below grey curve no toxicity was observed even at highest dose of beta-Peltatin used.

FIG. 4 shows dose-responses effects of beta-Peltatin used at different concentration (nM) for PPARd activation (black curve). The AC50 (upper part of the insert) and the maximum % activation (lower part of the insert) are given. Cellular toxicity was also assessed with standard method compared to untreated cells and shown in % of toxicity. As shown in the below grey curve no toxicity was observed even at highest dose of beta-Peltatin used.

### EXAMPLES

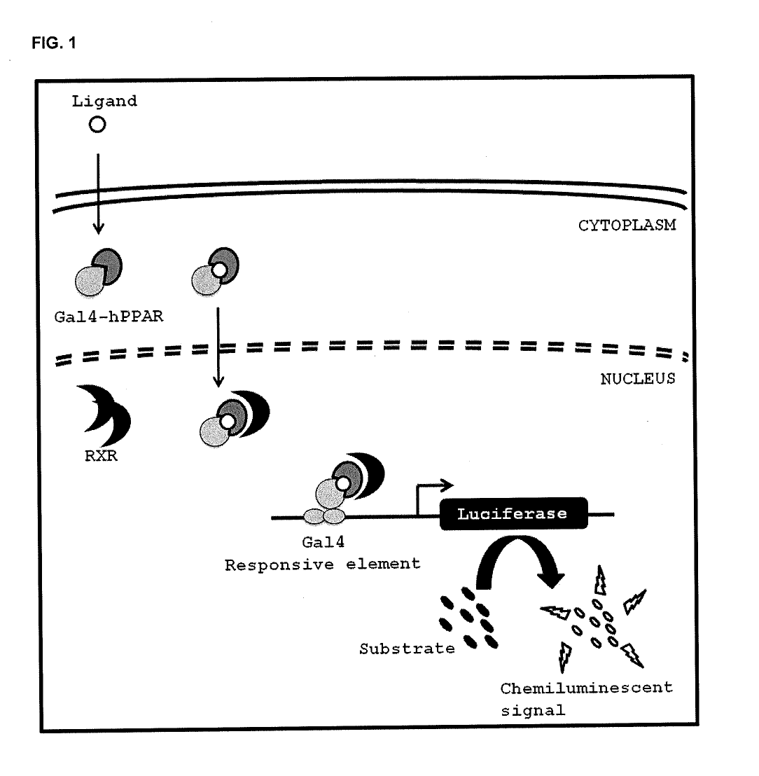

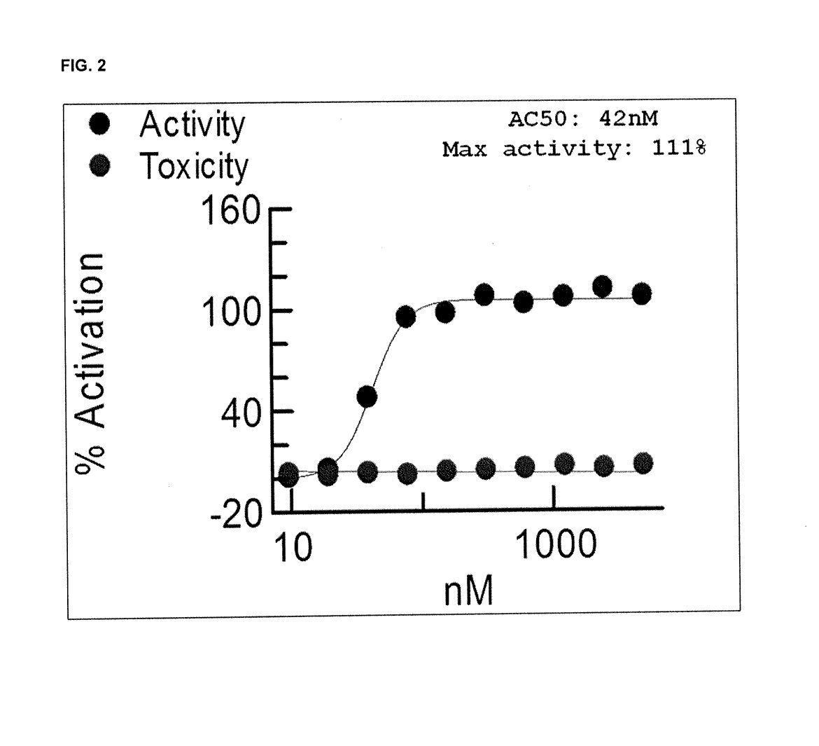

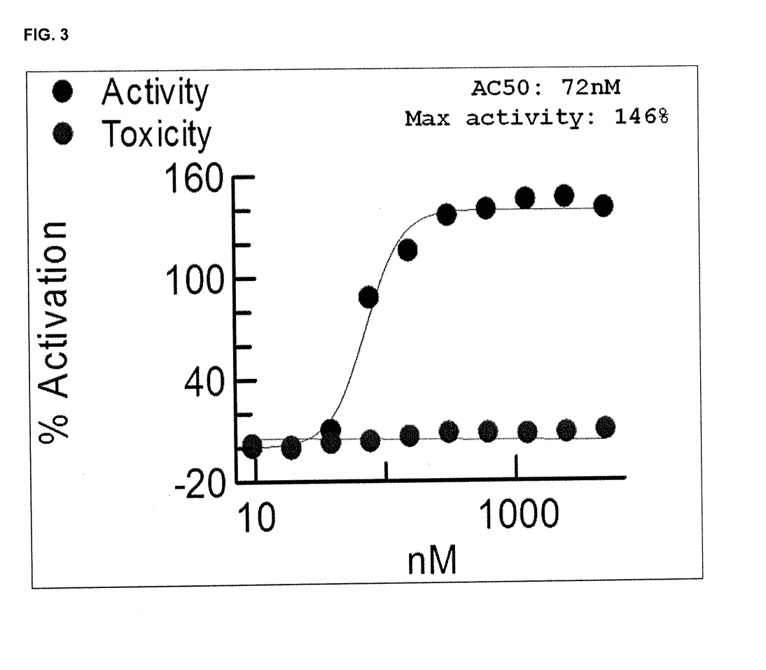

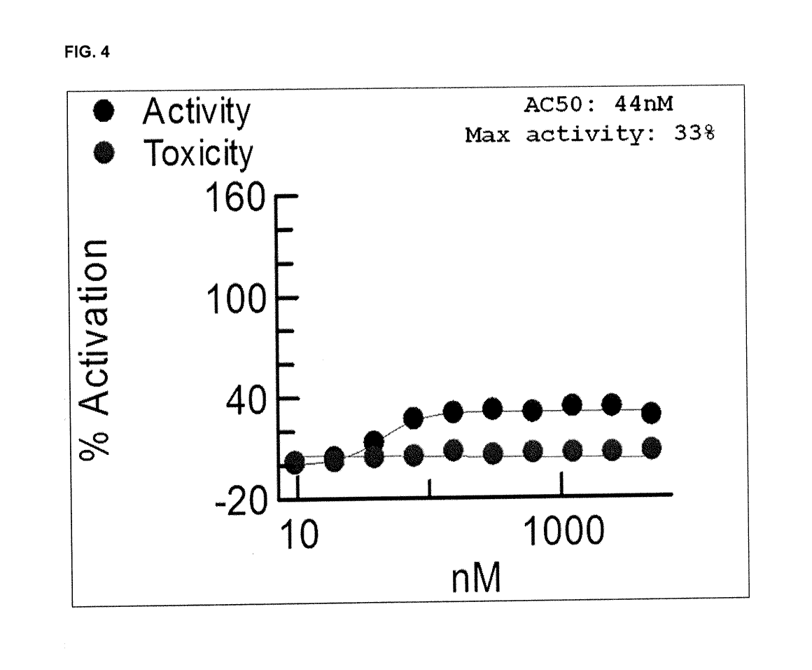

An edible plant fraction library was tested for PPARs agonist activity and toxicity. Then relevant plant fractions were further analyzed and bioactive(s) characterized and validated with the same hPPARa, hPPARg or hPPARd reporter bioassays. The reporter bioassay we used in order to identify anti-inflammatory plant properties targeted activation of human PPARs in human HeLa cells (as described in the FIG. 1). All isolated bioactives were tested in a dose response manner in duplicate. Assays were repeated 3-4 times. Shown are mean values obtained. Therefore specificity (for all the three known human PPAR receptors namely PPARα, PPARd and PPARg) and efficacy (full agonist i.e. % maximal activity >100% or partial agonist i.e. % maximal activity <100%) were tested (FIG. 2-4).

It was found that β-peltatin displayed dual agonistic activity on PPARα and PPARγ. It was further found that podophyllotoxin also displayed dual agonistic activity on PPARα and PPARγ.

PPARg nutrient sensing pathway is of high importance for cell metabolism, diabetes and inflammation and is supported by many published reports. Hence, pharma industries are already tackling PPARs pathways for many purposes. Peltatins or plant extracts containing peltatins might represent a novel and valuable nutritional approach for limiting inflammation by for instance limiting proinflammatory cytokine production.

1-14. (canceled)

15. A method of treating or preventing a chronic inflammatory disorder in a subject comprising administering a composition comprising at least one peltatin to the subject.

16. The method of claim 15, wherein the chronic inflammatory disorder is one which is treatable or preventable by activation of a peroxisome proliferator-activated receptor (PPAR).

17. The method of claim 15, wherein the chronic inflammatory disorder has a genetic or infectious origin.

18. The method of claim 15, wherein the chronic inflammatory disorder is selected from the group consisting of inflammatory bowel disease, Crohn's disease, ulcerative colitis, necrotizing enterocolitis, skin inflammation, inflammatory bowel syndrome, liver inflammation, bone inflammation, rheumatoid arthritis, systemic lupus, obesity-associated inflammation, and age-related low-grade inflammation.

19. The method of claim 15, wherein administering the composition alleviates inflammation.

20. The method of claim 15, wherein the peltatin is (i) α-peltatin or a derivative thereof, (ii) β-peltatin or a derivative thereof, or a combination of (i) and (ii).

21. The method of claim 15, wherein the peltatin is β-peltatin or a derivative thereof.

22. The method of claim 15, wherein the peltatin is provided as a plant extract.

23. The method of claim 22, wherein the plant extract is from Podophyllum peltatum or Anthriscus sylvestris.

24. The method of claim 15, wherein the composition is administered in an amount corresponding to about 0.01 to 100 mg dry weight peltatin/kg body weight.

25. The method of claim 15, wherein the composition is selected from the group consisting of food products, drinks, pet food products, cosmetic products, food additives, nutritional supplements, and powdered nutritional formulations to be reconstituted in milk or water.

26. The method of claim 15, wherein the composition is administered to teenagers, adults, or the elderly.

27. The method of claim 15, wherein the composition is administered immediately before or during a meal.

28. The method of claim 15, wherein the composition is administered with each meal.

29. The method of claim 15, wherein the composition comprises so amount of about 1-1000 mg peltatin per kg dry weight of the composition.

30. The method of claim 16, wherein the PPAR is PPARγ and/or PPARα.

31. The method of claim 15, wherein the subject is a human or an animal.

32. The method of claim 15, wherein the composition is administered enterally, parenterally, or topically.
